(19) United States
(12) Patent Application Publication
(10) Pub. No.: US 2026/0074062 A1
(43) Pub. Date: Mar. 12, 2026

(54) DEVICE FOR DETERMINING A LENGTH OF A CARDIAC ACTIVATION CYCLE

(71) Applicant: SUBSTRATE HD, Marseille (FR)

(72) Inventor: Dinara VESHCHEZEROVA, Marseille (FR)

(73) Assignee: SUBSTRATE HD, Marseille (FR)

(21) Appl. No.: 18/879,441

(22) PCT Filed: Jun. 29, 2023
(86) PCT No.: PCT/FR2023/051000

§ 371 Date: Nov. 17, 2025

(30) Foreign Application Priority Data
Jun. 30, 2022 (FR) FR2206690

(51) Int. Cl. G16H 40/63; A61B 5/00; A61B 5/346

(52) U.S. Cl. CPC G16H 40/63; A61B 5/346; A61B 5/7203

(57) ABSTRACT

A device for determining a cardiac activation cycle length including a memory arranged so as to store electrogram data having time stamps and associated with a channel. The device includes a preparer arranged so as to receive electrogram data associated with a given channel and with a time window of at least 1.5 seconds. The device includes a detector arranged so as to receive the pre-processed data and to detect therein non-overlapping activation segments that each correspond to a window within said time window of at least 1.5 seconds. The device includes a computer arranged so as to determine a periodicity condition of the activations by determining in each activation segment a reference time point and by comparing the duration of intervals each defined by two reference time points consecutive to the duration of the time window of at least 1.5 seconds.

### CROSS-REFERENCE TO RELATED APPLICATION(S)

This application is a 35 U.S.C. § 371 National Stage of International Patent Application No. PCT/FR2023/051000, filed on Jun. 29, 2023 claiming priority to and the benefit of French Patent Application No. 2206690, filed on Jun. 30, 2022, the disclosure of which is incorporated herein by reference in its entirety.

### TECHNICAL FIELD

The invention relates to the field of analysis of cardiac electrogram signals (hereinafter “EGM”).

### BACKGROUND

Cardiac electrograms are obtained by introducing catheters into the heart of a person and by measuring the cardiac signals thanks to these.

The length of the cycle (or “CL” for “Cycle Length”) is a measurement used for characterising the electrical activity in the atria. In particular, this characterisation is used to guide practitioners during catheter ablation operations. It is measured in milliseconds and generally reflects the time for which a complete relaxation and contraction cycle of the atria (or ventricles, or both) occurs.

For doctors, it is essential to monitor two values: an overall CL of the atria—recorded from the stable reference catheter—and a local CL (or “LCL” for “Local Cycle Length”)—characteristic of the electrical activity under the mapping catheter which differs from the overall CL in the pathological substrate. Next, the expression CL will be used to qualify the overall CL, and the expression LCL to qualify the CL of the mapping catheter.

During an atrial fibrillation (or “AF”), there is no common wavefront originating from the sinus node, but several wavefronts reflecting the propagation of the potential in different portions of the atria according to the electrical remodelling of the cardiac tissue. For example, recent searches have shown that the areas where the =catheter ablation has allowed putting an end to a persistent AF feature a fast and organised activity. For these reasons, the estimation of the LCL is very important to help find these areas quickly. The relative difference between the LCL and CL length values allows judging the mapped area. Ideally, the clinician should have access to a reliable estimate of the LCL, at a refresh rate sufficient for an optimum clinical workflow.

This estimation is extremely difficult to carry out. Indeed, the local electrical activity is often generated by several neighbouring sources having different characteristics, which gives EGM characterised by a great variability both in terms of amplitude and waveforms, which makes the LCL analysis more difficult. Furthermore, the mapping catheter is not fixed and moves a lot during the catheter ablation, which means that the signals are substantially influenced by the noise added by the different sources and the far-field activity during the contactless phases.

Current solutions generally belong to two families. The first family is based on conventional signal processing methods, for example based on fast Fourier transform or on the study of the autocorrelation which is a conventional method used to assess the length of the cycle for the periodic signals of any kind. The second family comprises different adaptive thresholding methods based on the detection of auricular activations by the amplitude. These methods form the recent trend in the field of computational cardiology research.

Some of these methods will now be described. All propose a specific pre-processing, but the latter is almost systematically based on the pre-processing introduced in the article by Botteron and Smith “A technique for measurement of the extent of spatial organisation of atrial activation during atrial fibrillation in the intact human heart”, IEEE Transactions on Biomedical Engineering, vol. 42, no. 6, pp. 579-586, 1995. This pre-processing begins with the application of a band-pass filter of 40-250 Hz to accentuate the signal corresponding to the local depolarisation, followed by a rectification of the resulting signal to take account of the biphasic nature of the bipolar recordings, and ends with the application of a low-pass filter with a cut-off at 20 Hz to limit the spectrum to the frequencies included in a reasonable physiological range of activation rate, the frequency rate of the AF being normally comprised between 4 and 10 Hz. This technique allows obtaining a signal whose waveforms are proportional to the amplitude of the initial components of the EGM with frequencies within the interval of the cut-offs of the band-pass filter.

The method described in the article by Everett et al. “Frequency domain algorithm for quantifying atrial fibrillation organisation to increase defibrillation efficacy” IEEE Transactions on Biomedical Engineering, vol. 48, no. 9, pp. 969-978, 2001 belongs to the first family and suggests transforming the envelope of an input EGM using fast Fourier transform (FFT). Before applying it, a Botteron pre-processing is performed at first. Afterwards, in order to attenuate discontinuities at the beginning and at the end of a segment, windowing with a rounded waveform like that of Hanning or Kaiser is generally applied. The resulting signal is subjected to a Fourier transform. The obtained power spectrum generally has a maximum peak in the frequency range from 3 Hz to 20 Hz, so-called dominant frequency. The dominant frequency value corresponds approximately to the length of the auricular activation cycle.

In the thesis “Analysis of Atrial Electrograms” by Christopher Schilling, Vol 17 Karlsruhe Transactions on Biomedical Engineering, Karlsruhe Institute of Technology (KIT) Institute of Biomedical Engineering, a broad and meticulous analysis of atrial EGM is proposed. The author describes therein a segmentation of the electrograms into active and inactive portions in the interval received at the input. The proposed algorithm is based on the Pan-Tompkins QRS detection algorithm described in the article “A real-time QRS detection algorithm”, IEEE Transactions on Biomedical Engineering, pp. 230-236, 1985. Instead of directly transmitting the EGM received at the input of the algorithm, the non-linear energy operator (NLEO) is used on the basis of the theory described by Kaiser et al. in the article “On a simple algorithm to calculate the ‘energy’ of a signal”, Acoustics, Speech, and Signal Processing, 1990. ICASSP-90, 1990 International Conference on, pp. 381-384, 1990, which describes the NLEO as a simple calculation of the energy of discrete signals over time which preserves the amplitude as well 10 as the frequency of an input signal.

After a pre-processing to remove baseline noise, the NLEO operator focuses on the high-frequency and high-amplitude sections. The output is low-pass filtered in order to smooth the signal with a Gaussian sliding window. The effective width of the impulse response as well as the cut-off frequency of the filter are specifically selected according to the properties of the activations of the AF. The non-zero portions of the obtained signal are considered to be active. To detect them, the author proposes an adaptive thresholding method based on the standard deviation (weighted by a factor k=0.1) calculated for a set of overlapping windows. This portion aims to highlight the high peaks and ignore the low peaks. Using a sliding window of 1 s 25 with a step of 50 ms, each point of the input signal obtains 20 thresholds (a standard value for 20 windows). For each point, the minimum standard is selected as the final threshold. Finally, since the difference between two active segments should be longer than 42 ms—a value 30 related to the average duration of the refractory period
the shorter inactive segments are discarded and the neighbouring active segments are merged. According to doctors, the active segments of less than 10 ms have no physiological significance and are marked as inactive during this post-processing. This method is insufficiently accurate and requires a particular adaptation to each person, according to its AF.

The article by Osorio et al. “Comparative Study of Methods for Atrial Fibrillation Cycle Length Estimation in Fractionated Electrograms”, IEEE Computing in Cardiology (CinC), 2017, describes and compares three methods with an adaptive threshold. The first one is an amplitude threshold adaptation method described in the article by Faes L. et al. “A method for quantifying atrial fibrillation organisation based on wave-morphology similarity”, IEEE Transactions on Biomedical Engineering 2002; 49(12 I): 1504-1513. The second one is an iterative CL method described in the article by Ng J, and al. “Iterative method to detect atrial activations and measure cycle length from electrograms during atrial fibrillation”. IEEE Transactions on Biomedical Engineering 2014; 61(2): 273-278. Finally, the third is a hybrid fractionation degree method (or “FHD” standing for “hybrid fractionation degree”) described in the article by Osorio et al. “A fractionation-based local activation wave detector for atrial electrograms of atrial fibrillation”, in Computing in Cardiology Conference (CinC), volume 44. IEEE, 2017; In press. Herein again, these methods do not work in a satisfactory manner with a mapping catheter.

Finally, the article by Milad El. Haddad et al. “Algorithmic detection of the beginning and end of bipolar electrograms: Implications for novel methods to assess local activation time during atrial tachycardia”, Biomedical Signal Processing and Control, Volume 8, Issue 6, November 2013, Pages 981-991, describes a method for detecting the start and the end of each active complex presented in a bipolar EGM sample. The two main steps of the developed algorithm are: 1) detecting the start time point and the end time point of the activation complex using the reference catheter to identify a mapping window where the search is performed, and 2) determining the local activation time (or “LAT”) by three different methods, as well as the signal-to-noise ratio (SNR). This method also does not give satisfaction with a mapping catheter.

None of these methods, nor others of the first family or of the second family, is satisfactory, with regards to their inability to manage cardiac signals that are too different as well as to their unsuitability to the particular case of AF and/or use with a mapping catheter.

### SUMMARY OF THE INVENTION

The invention improves the situation. To this end, it provides a device for determining a cardiac activation cycle length comprising a memory arranged so as to store electrogram data having time stamps and associated with a channel, a preparer arranged so as to receive electrogram data associated with a given channel and with a time window of at least 1.5 seconds, to extract therefrom baseline noise and high-frequency noise and to provide pre-processed data, a detector arranged so as to receive the pre-processed data and to detect therein non-overlapping activation segments that each correspond to a window within said time window of at least 1.5 seconds, which detector operates by determining local extrema in the pre-processed data and by grouping them into activation segments, and a computer arranged so as to determine a periodicity condition of the activations by determining in each activation segment a reference time point and by comparing the duration of intervals each defined by two reference time points consecutive to the duration of the time window of at least 1.5 seconds, and, in case of a periodicity condition indicating periodic activation segments, to determine an activation cycle length from the average or the median of the interval durations.

This device is particularly advantageous because it allows determining a LCL value which adapts to numerous cardiac contexts involving AF. In addition, this device is most particularly suited to use during a catheter ablation procedure.

According to various embodiments, the invention may have one or more of the following features:
the computer is further arranged so as to determine the periodicity condition of the activations from the ratio of the difference between the longest interval duration and the shortest interval duration divided by the average of the duration of the intervals,
the detector is arranged so as to calculate the ratio of the difference between the longest interval duration and the shortest interval duration divided by the average of the duration of the intervals, and to redetect the activation segments by modifying the detection of the extrema when this value exceeds a selected threshold,
the detector is arranged so as to perform a post-processing by cutting into two or more the activation segments whose duration is longer than twice the average of the durations of the activation segments,
the detector is arranged so as to perform a post-processing by segmenting the intervals into three groups according to their duration, by searching for local extrema in the electrogram data corresponding to the intervals of the group of longest durations, and, where appropriate, by completing the activation segments with activation segments derived from these extrema,
the detector is arranged so as to perform post-processing by segmenting the intervals into three groups according to their duration, and by merging the activations segments whose reference time points define the intervals of the group of shortest durations,
the detector is arranged so as to calculate the ratio of the difference between the longest interval duration and the shortest interval duration divided by the average intervals of the intervals before and after post-processing, and to apply the post-processing only when this ratio decreases, and
the computer is arranged so as to determine an agglomerated cycle length value from the cycle length value of several electrodes with reference to the same time window.

The invention also relates to a method for determining a cardiac activation cycle length comprising:
a) receiving electrogram data having time stamps and associated with a given channel and with a time window of at least 1.5 seconds,
b) extracting from the electrogram data of the operation a) baseline noise and high-frequency noise to provide pre-processed data,
c) detecting in the pre-processed data non-overlapping activation segments each corresponding to a window within said time window of at least 1.5 seconds, by determining local extrema in the pre-processed data and grouping them into activation segments,
d) determining a periodicity condition of the activations by determining in each activation segment a reference time point and by comparing the duration of intervals each defined by two reference time points consecutive to the duration of the time window of at least 1.5 seconds, and, in case of a periodicity condition indicating periodic activation segments, determining an activation cycle length from the average or the median of the durations of intervals.

According to various embodiments, this method may have one or more of the following features:
the operation d) further comprises determining the periodicity condition of the activations from the ratio of the difference between the longest interval duration and the shortest interval duration divided by the average of the duration of the intervals.
the operation c) comprises c1) calculating the ratio of the difference between the longest interval duration and the shortest interval duration divided by the average of the duration of the intervals, and c2) re-detecting the activation segments while modifying the detection of the extrema when this value exceeds a selected threshold.
the operation c) comprises one or more of the following post-processing operations:
c3) cutting into two or more than two the activation segments whose duration is longer than twice the average of the durations of the activation segments,
c4) segmenting the intervals into three groups according to their duration, by searching for local extrema in the electrogram data corresponding to the intervals of the group of longest durations, and, where appropriate, by supplementing the activation segments with activation segments derived from these extrema,
c5) segmenting the intervals into three groups according to their duration, and by merging the activation segments whose reference time points define the intervals of the group of shortest durations, and
   the operation c) further comprises c6) calculating the ratio of the difference between the longest interval duration and the shortest interval duration divided by the average duration of the intervals before and after post-processing, and applying the post-processing only when this ratio decreases.

The invention also relates to a computer program comprising instructions for executing the method according to the invention, a data storage medium on which such a computer program is recorded and a computer system comprising a processor coupled to a memory, the memory having such a computer program recorded therein.

### BRIEF DESCRIPTION OF THE DRAWINGS

Other features and advantages of the invention will appear more clearly upon reading the following description, derived from examples given for illustrative and non-limiting purposes, from the drawings wherein:

FIG. 1 shows a generic diagram of a device according to the invention,

FIG. 2 shows an example of an operation loop of the device of [FIG. 1],

FIG. 3 shows an example of implementation of a function implemented in an operation of [FIG. 2],

FIG. 4 shows an example of implementation of a function implemented in an operation of [FIG. 3], and

FIG. 5 shows an example of implementation of a function implemented in an operation of [FIG. 2].

### DETAILED DESCRIPTION

The drawings and the description hereinafter essentially contain elements of certain nature. Hence, they could not only be used to better understand the present invention, but also contribute to definition thereof, where appropriate.

The present description could involve elements likely to be protected by copyright. The owner of the rights has no objection with regards to the identical reproduction by anyone of the present patent document or its description, as it appears in official records. Otherwise, it completely reserves its rights.

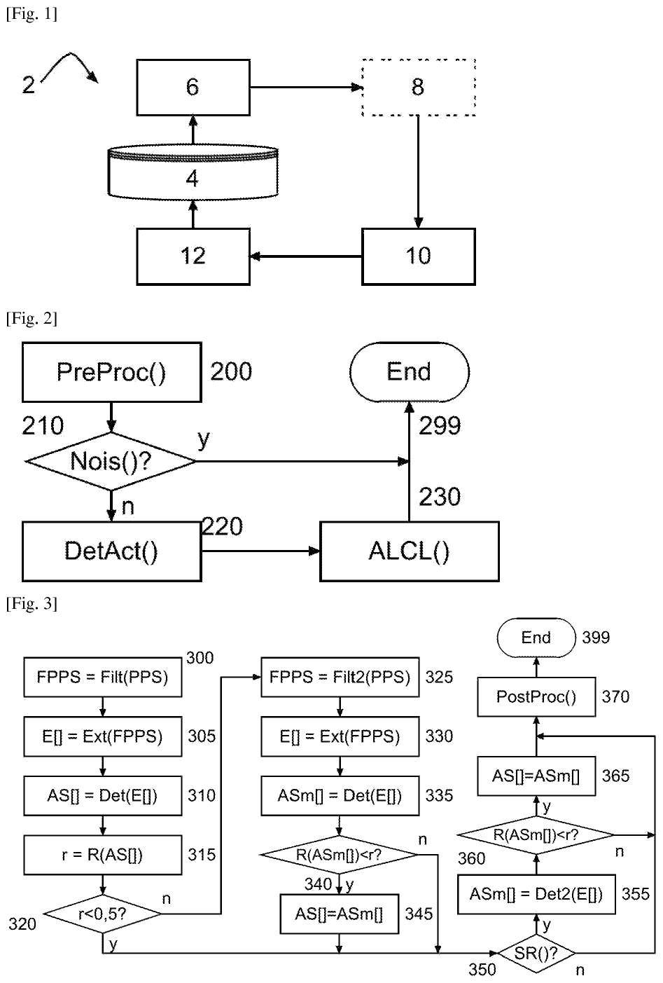

FIG. 1 shows a generic diagram of a device 2 according to the invention. The device 2 comprises a memory 4, a preparer 6, a filter 8, a detector 10 and a computer 12. As it will be seen hereinbelow, the filter 8 is optional.

The memory 4 receives the data which are subjected to functions and calculations implemented by the device 2. The device 2 primarily processes electrogram data, i.e. electrical signals measured by a multi-electrode catheter during an intracardiac procedure. These signals are generally digital, and each sample has an amplitude value and is associated on the one hand with a time stamp and, on the other hand, with an electrode identifier through which it has been obtained. Other data may be associated with the electrogram data, but these data are sufficient to define what is called channels, i.e. a reading of electrical measurements referenced over time. The electrode identifier allows taking account of the fact that the measurements are generally carried out with several channels simultaneously, and distinguishing them. The catheters may be unipolar or bipolar or according to any other technology. As it will be seen hereinbelow, these data are denoised and then transformed in order to determine activation segments, which represent continuous time portions during which a practitioner considers that the electrogram data indicate an activation of the cardiac tissue under the electrode. These activation segments are conveniently defined by a start time stamp and an end time stamp. Next, an activation segment will designate both the time range corresponding to these time stamps and the corresponding electrogram data, for the considered electrode identifier.

The memory 4 may consist of any type of data storage capable of receiving digital data: hard disk, flash memory hard disk, flash memory in any form, random-access memory, magnetic disk, a storage distributed locally or in the cloud, etc.

In the example described herein, the memory 4 receives all of the data described hereinabove, and more generally all of the data that relate to the device 2, i.e. the programs and software instantiating the preparer 6, the filter 8, the detector 10 and the computer 12, their parameters, the data received at the input, the data at the output, as well as the data stored in the buffer memory. The data calculated by the device may be stored on any type of memory similar to the memory 4, or on the latter. These data may be erased after the device has performed its tasks or kept.

The preparer 6, the filter 8, the detector 10 and the computer 12 directly or indirectly access the memory 4. They may be made in the form of an appropriate computer code executed on one or more processor(s). By processors, it should be understood any processor adapted to the calculations described hereinbelow. Such a processor may be made in any known manner, in the form of a microprocessor for a personal computer, a laptop, a tablet or a smartphone, an FPGA or SoC type dedicated chip, a computing resource on a grid or in the cloud, a cluster of graphics processors (GPUs), a microcontroller, or of any other form capable of providing the computing power necessary for the embodiment described hereinbelow. One or more of these elements may also be made in the form of specialised electronic circuits such as an ASIC. A combination of a processor and electronic circuits may also be considered. In the case of the machine learning unit based on gradient reinforcement, processors dedicated to machine learning could also be considered.

The preparer 6, the filter 8, the detector 10 and the computer 12 are described herein separately because they carry out distinct functions. This modular description aims to better understand the functional blocks implemented by the device 2. It goes without saying that two or more of these elements could nevertheless be grouped insofar as the functional relationships remain comparable.

FIG. 2 shows an example of an operation loop of the device of the [FIG. 1] allowing better understanding the respective functions of the preparer 6, of the filter 8, of the detector 10 and of the computer 12.

Next, the electrogram data are processed by a time window of 1.5 seconds. Alternatively, this time window could be wider. Thus, next, the expression “a signal” or “a channel” will designate the electrogram data associated with a time window of 1.5 seconds and with a given electrode identifier. In addition, the following description is given for a channel. Nevertheless, several synchronised channels may be processed in parallel, which, as it will be seen hereinbelow, allow determining an agglomerated LCL value.

In a first operation 200, the device 2 calls a function PreProc( ) which is executed by the preparer 6. The function PreProc( ) receives as argument a channel and returns a pre-processed signal which will be referenced PPS hereafter. In the example described herein, the pre-processing operations comprise, on the one hand, the suppression of baseline noise (“baseband wander removal”) by means of a Butterworth filter, whose critical frequency is 10 Hz and, on the other hand, the suppression of high-frequency noise using the discrete wavelet transform thresholding method described in the article by Donoho et al. “De-noising by soft-thresholding”, IEEE Transactions on Information Theory, vol. 41, no. 3, pp 613-627, May 1995, doi: 10.1109/18.382009. Although these operations are preferred by the Applicant, other pre-processing operations could supplement them or replace them, like those of the article by Botteron cited before.

Afterwards, in an optional operation 210, the filter 8 executes a function Nois( ) which analyses the PPS signal to determine whether it is exploitable or it is too noisy. In the example described herein, the function Nois( ) applies three successive filters: a kurtosis filter for analysing the PPS signal, a motion detection filter and an amplitude filter.

The cardiac signal is essentially a noisy isoelectric line with several more or less neat single-phase or multi-phase waves which represent passages of electric waves under the electrode. Hence, it is expected that the distribution of the values is centred around zero, but with a significant tail (the values in the tail represent the potential related to the activations, when the signal corresponds to a periodic cardiac activity). To describe this probability distribution shape, a statistical measure of the kurtosis is conventionally used. The kurtosis identifies whether the tails of a given distribution contain extreme values. If the kurtosis is lower than 2.5, or negative, then the signal is considered to be noise and is discarded. Alternatively, this filter may be ignored, a different threshold may be selected or a method other than kurtosis may be used.

The motion detection filter aims to detect a change in activity related to the motion, to the contact/non-contact phases or to the insertion of the catheter. For this purpose, the signal is divided into three portions of 0.5 seconds, the variance of each portion is calculated and the ratio of the highest variance to the lowest one is compared with a threshold value in the range of 10\hat{ }6. This threshold has been identified as particularly advantageous by the Applicant during its works. Alternatively, this filter may be ignored, another threshold may be retained or another method may be retained.

The amplitude filter aims to reflect the fact that the extremely low voltage signals—invisible during the procedure—mainly represent noise. For this purpose, the channel is thresholded at 0.03 mV, i.e. all of the amplitudes lower than this threshold are brought back to 0. Similarly, the signals with a high amplitude are characteristic of a high noise during the insertion of the catheter or the saturated stimulation schemes. For this reason, the channel is also thresholded at 3.6 mV, i.e. all of the amplitudes higher than this threshold are brought back to 3.6 mV. Alternatively, this filter may be ignored or the thresholds modified.

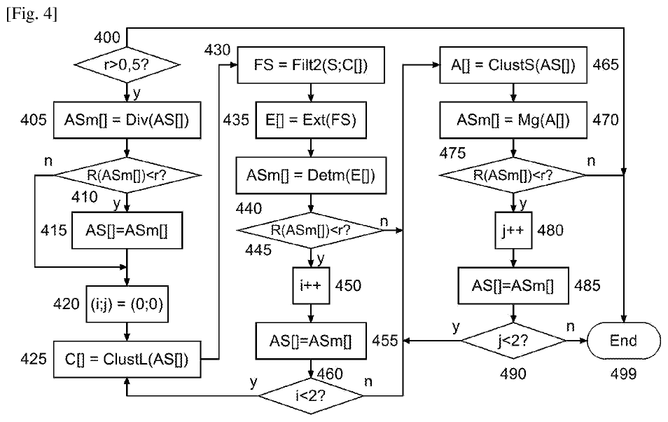

Afterwards, the Signal PPS (denoised or not) is processed in an operation 220 by the detector 10 for the main the operation of the device 2: the detection of activation segments. The aim is to identify in the channel which portion corresponds to a cardiac activity. For this purpose, the detector 10 executes a function DetAct( ) an example of implementation of which will be described by means of FIGS. 3 and 4.

Once the activation segments have been detected, the computer 12 executes a function ALCL( ) in an operation 230 in order to determine a LCL value for the channel if this is possible, as well as an agglomerated LCL value when several channels are processed and their respective LCL values enable so.

Finally, the loop ends in an operation 299, and the device 2 can process one or more channel(s) corresponding to a subsequent window.

FIG. 3 shows an example of implementation of the Function DetAct( ) In an operation 300, the detector 10 executes a function Filt( ) which carries out an adaptive thresholding of the PPS signal. For this purpose, the function Filt( ) analyses the entire signal and keeps only the data whose amplitude value in absolute value is beyond the 95th percentile, i.e. the 5% of the highest values. The other values are brought back to 0. The resulting signal is designated by the reference FPPS (standing for “filtered pre-processed signal”).

Afterwards, in an operation 305, a function Ext( ) is executed on the signal FPPS to detect local extrema therein. Because of the previous operation 300, the detected extrema correspond to the non-noisy peaks of the initial signal. Numerous methods are known to a person skilled in the art to identify these local extrema, which are returned as output in a table E[ ].

Once the extrema have been determined, a function Det( ) is executed in an operation 310 to identify activation segments. The general idea is that an activation segment is a portion of the signal which comprises relatively close extrema which may be separated by zero values, and such that between two distinct activation segments, all of the values of the signal are zero. Consequently, the function Det( ) uses, in the example, a sliding window for which, at each time point, the number of extrema in the window is associated. This allows carrying out an estimation of the density of the extrema. By construction, between two segments, the value is zero—since all values are zero. This allows delimiting the bounds of the activation segments which are stored in a table AS[ ]. In the example described herein, the table AS[ ] comprises, for each segment, the start time stamp and the end time stamp, which are sufficient to identify all of the electrogram data or corresponding or PPS signal or FPPSs. Alternatively, these data could be stored in the table AS[ ]. The function Det( ) also determines a reference time point for each activation segment. In the most preferred variant, this time point is that one for which the signal has the highest absolute value in the signal FPPS. Alternatively, it could be selected differently, for example by being the start time stamp or the end time stamp of the activation segment, in a state determined by a barycentre-type formula of the non-zero values of the electrogram data of the considered activation segment, or by averaging the time stamps of a restricted number of electrogram data of the activation segment having the highest absolute value (for example the three highest values).

In a preferred variant of the invention, the operations 300, 305 and 310 may be replaced by a detection of local extrema according to which an extremum is kept only if it belongs to the 95th percentile. Afterwards, a normal kernel (Gaussian window) is placed at the centre of each extremum. The superposed kernels are added, giving an estimate of the density of the smooth kernel. Hence, the obtained density represents the distribution of the extrema over the signal and therefore allows finding the bounds of the activation segments by targeting infinitesimal areas of the density. This corresponds to a kernel density estimation algorithm, as described for example at the address
https://web.archive.org/web/20220414175413/https://en.wikipedia.org/wiki/Kernel_density_estimation

Afterwards, a function R( ) is executed on the table of activation segments AS in an operation 315. The reference time points of each activation segment form the intervals two-by-two. The Applicant has discovered that these intervals are of great importance, and that it is in particular crucial that they have some periodicity and resemblance with one another.

The function R( ) calculates a ratio r according to the following formula:

$$r = ( max ( i ) - min ( i ) ) / mean ( i )$$

where max(i) and min(i) are respectively the longest and shortest interval duration among the intervals defined by the reference time points of the table AS[ ], and mean(i) is the average of the duration of the intervals defined by the reference time points of the table AS[ ]. The value r is a variability value which is robust to the amplitude in absolute value and does capture the spread of the values. The Applicant has observed that a value of r of 0.5 is particularly discriminating for the obtainment of reliable results in the case of the AF. Alternatively, this value could be comprised between 0.35 and 0.75. The ratio r is a measure of the variability and of the extent of the values. Although the formula set out herein is preferred, other formulas could be retained to assess this ratio, insofar as they capture the measurement of the variability and of the extent of the values.

When the ratio r is higher than 0.5, this means that the intervals defined by the activation segments are not similar enough. One of the causes might be the function Filt( ). For this reason, the operations 300, 305 and 310 are repeated in the operations 325, 330 and 335, except that the operation 325 executes a function Filt2( ) in which the threshold is set to the 90th percentile. Alternatively, the threshold could be set lower than the 90th percentile. An operation 340 tests whether the ratio obtained by these operations is lower than in the operation 320. If so is the case, then the table ASm[ ] of the operation 335 replaces the table AS[ ] in an operation 340.

After the operation 340, or when the ratio r is lower than 0.5 in the operation 320, or when the operations 325 to 335 do not allow improving the ratio r, a test is carried out in an operation 350 in order to determine whether the channel is characteristic of a slow rhythm. For this purpose, a function SR( ) is executed and tests whether more than 5 activation segments have been found in the channel. Indeed, if so is not the case, then the LCL value is potentially greater than 300 ms. Although this is possible, this may also be due to the various denoising operations or to the influence of the far field. For this reason, when a slow rhythm is detected, the operation 310 is repeated in an operation 355 but with a sliding window with an increased width. In this same operation, the segments in which the amplitude is lower than 90% of the maximum amplitude of the channel are suppressed. An operation 360 tests whether this improves the ratio r, and, if so is the case, the table AS[ ] is replaced in an operation 365 by the table ASm[ ] produced by the operation 355.

Finally, the function DetAct( ) launches in an operation 370 a function PostProc( ) then the function DetAct( ) ends with an operation 399.

The operations 350, 355, 360 and 365 are optional, and the operations 320, 340 and 345 could directly lead to the operation 370.

An example of implementation of the function PostProc( ) will now be described with reference to [FIG. 4]. The principle of the function PostProc( ) is as follows:
the post-processing targets a ratio r lower than 0.5,
each processing is repeated at most three times, and each iteration is accepted only if it improves the ratio r,
first of all, it is sought to cut the activation segments that are too long, then an attempt is made to work on the intervals.

Thus, the function PostProc( ) starts in an operation by an operation 400 by a test on the ratio r. Indeed, if the function PostProc( ) is called after the operation 320, then the ration r is necessarily lower than 0.5 and no post-processing is necessary. If so is the case, then the function ends in an operation 499.

If not, a first post-processing is executed in the operations 405, 410 and 415 to cut the activation segments that are too long. Thus, in the operation 405, a function Div( ) is executed by the detector 10 in order to cut into two any activation segment, whose duration is longer than twice the average of the duration of all of the activation segments. The operation 410 tests whether this operation improves the ratio r, and, if so is the case, the table AS[ ] is replaced in the operation 415 by the table ASm[ ] produced by the operation 405.

After this first post-processing, indices i and j are initialised at 0 in an operation 420. These indices allow ensuring that the following two post-processings are not repeated more than 3 times.

The second post-processing comprises operations 425, 430, 435, 440, 445, 445, 450, 455 and 460. In the operation 425, a function ClustL( ) is executed on the table AS[ ]. This function is intended to segment the intervals of the table AS[ ] into three groups: a group of “short” intervals, a group of “average” intervals, and a group of “long” intervals. The philosophy behind this post-processing is that the group of “average” intervals is supposed represent intervals that do not need post-processing, unlike the group of “short” intervals and the group of “long” intervals. This second post-processing covering the groups of “long” intervals, the function ClustL( ) returns a table C[ ] which contains all of the intervals of the group of “long” intervals. In the example described herein, this segmentation is carried out by means of a jenkspy programming library (cf. the address the https://pypi.org/project/jenkspy/), implements which the Fisher-Jenks natural break algorithm (cf. for example the address https://web.archive.org/web/20220617141817/https://en.w ikipedia.org/wiki/Jenks_natural_breaks_optimization). This algorithm has the advantage of being unsupervised. Other algorithms (kmeans, etc.) could be used.

Afterwards, in an operation 430, the original channel signal S is re-processed in an operation 430 to try to detect activations that would have been missed within the intervals of the table C[ ]. The idea herein is that the various denoising operations have allowed smoothing some extrema, which has produced the intervals that are too long. For this purpose, the operation 430 executes the function Filt2( ) but on the channel signal S before the operation 200, and only on these intervals. The operation 435, identical to the operations 305 and 330, executes the function Ext( ) in order to determine the extrema in the signal FS resulting from the operation 430. Afterwards, the resulting table E[ ] is processed in the operation 440 in a manner similar to the operations 310 and 335, with the execution of a function Detm( ) similar to the function Det( ) but which replaces the activation segments of the table AS[ ] corresponding to the intervals of the table C[ ] by those found by the function Detm( ) and stores all in the table ASm[ ]. Like before, the aim is to reduce the value of the ratio r, which is verified in the operation 445. If so is the case, then index i is incremented in the operation 450, and the table AS[ ] is replaced by the table ASm[ ] in the operation 455. Finally, in the operation 460, the index i is tested to limit to 3 the repetition of the operations 425 to 455, and, if so is the case, then the loop of the second post-processing resumes with the operation 425. If so is not the case, or if the test of the operation 445 is negative, then the third post-processing is launched.

The third post-processing relates, as indicated hereinabove, to the groups of “short” intervals. For this purpose, operations 465, 470, 475, 480 and 485 are repeated up to three times, as long as the ratio r is improved.

Thus, the segmentation of the operation 425 is repeated in the operation 465, but with a function ClustS( ) which returns a table A[ ] containing the intervals of the group of “short” intervals instead of the table C[ ]. Afterwards, in the operation 470, a function Mg( ) tests the interval of the table A[ ] and verifies whether its duration multiplied by 1.75 is longer than the duration of the longest of the intervals of the group of “average” intervals. If so is the case, this means that the intervals of the table A[ ] are not sufficiently distinct from those of the group of “average” intervals and should not be the subject of post-processing. If so is the case, then the function Mg( ) modifies the table AS[ ] in order to merge, for each interval of the table A[ ], the two activation segments whose respective reference time points serve as a bound to an interval, and the result is stored in the table ASm[ ]. Like before, the aim is to reduce the value of the ratio r, which is verified in the operation 475. If so is the case, then the index j is incremented in the operation 480, and the table AS[ ] is replaced by the table ASm[ ] in the operation 485. Finally, in the operation 490, the index j is tested to limit to 3 the repetition of the operations 465 to 485, and, if so is the case, then the loop of the second post-processing resumes with the operation 465. If so is not the case, or if the test of the operation 475 is negative, then the third post-processing is completed and the function PostProc( ) ends in the operation 499.

Numerous variants may be considered for the function PostProc( ) Thus, one or two of the three posttreatments could be omitted, their order could be modified, although the Applicant has discovered that this order offers the best results. In addition, the second and third post-processings could be carried out simultaneously in order to gain in speed. In addition, the parameters of these post-processings, like the ratio thresholds or the number of iterations, could vary.

In the functions DetAct( ) and PostProc( ), numerous retreatments are conditioned by a decrease in the ratio r. In some variants, this condition could be omitted.

Once the Function DetAct( ) is completed, the table AS[ ] therefore comprises all of the detected activation segments, as well as the corresponding reference time points which define the intervals.

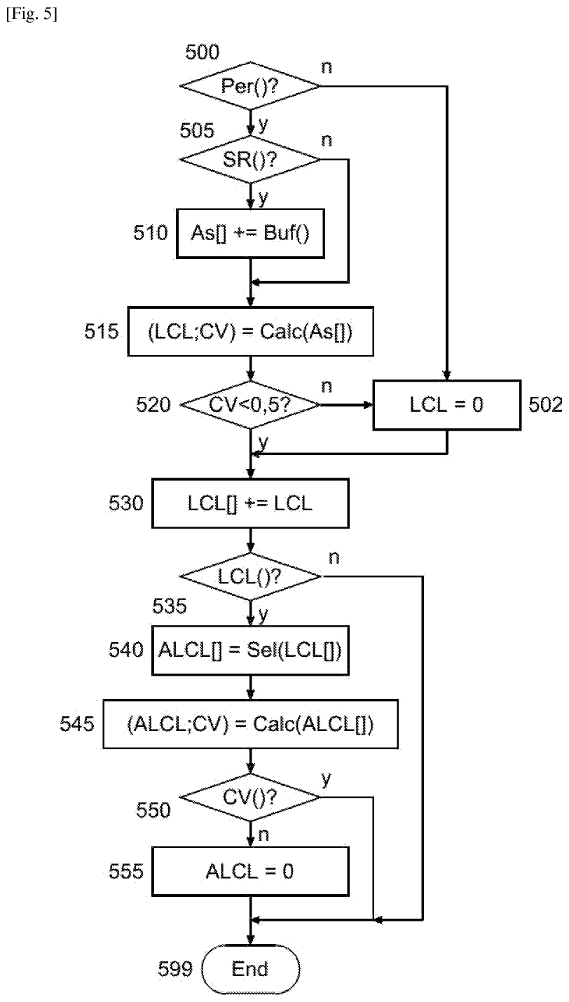

FIG. 5 shows an example of implementation of the function ALCL( ) implemented by the computer 12 to estimate the LCL value and possibly an agglomerated LCL value.

The function ALCL( ) comprises two portions. In a first portion, this function calculates the LCL value for the channel that has just been processed, then it seeks to determine whether an agglomerated LCL value could also be calculated.

The first portion begins with an operation 500 in which a function Per( ) is executed. The function Per( ) aims to determine whether the intervals defined by the activation segments define a periodic activation. For this purpose, in the example described herein, the function Per( ) tests three conditions:
the duration of each interval is calculated in order to verify that none of them exceeds one-quarter of the channel,
the total duration of the activation segments is measured to verify that it occupies more than half the channel, and
the ratio r is recalculated to verify that it is lower than 0.6.

If one of these three conditions is not met, then the channel is considered to be unexploitable and the LCL value is set to 0 in an operation 502.

Otherwise, the channel is considered to be periodic, and an optional operation 505 verifies whether the rhythm of the channel is slow or not. If so is the case, then in an operation 510 that is also optional, the table AS[ ] is completed with prior table data AS[ ] in a buffer memory in order to have enough activation segments to be able to calculate the LCL value.

Afterwards, in an operation 515, a function Calc( ) determines from the table AS[ ] a LCL value as well as a value CV which represents the variance of the interval durations within the table AS[ ]. In the example described herein, the LCL value is obtained by determining the median of the durations of the intervals in the table AS[ ]. Alternatively, the average or a value derived from the median, the average or otherwise barycentric could be used. In the example described herein, the value CV is obtained by dividing the standard deviation of the interval durations by the average of these.

The value CV is then tested in an operation 520 in order to determine whether the intervals are homogeneous enough with one another. If this value exceeds 0.5, then it is considered that this is not the case, and the LCL value is set to 0 in the operation 502.

Finally, the first portion of the function ALCL( ) ends in an operation 530 in which the LCL value is introduced into a table LCL[ ]. The value “0” indicates that the LCL value is either associated with a non-periodic channel, or insufficiently homogeneous.

When the table LCL[ ] receives all of the LCL values of each channel for a given period of time, the function ALCL( ) may in the second portion attempt to determine an agglomerated LCL value.

Thus, in an operation 535, a function LCL( ) tests the table LCL[ ] to determine whether it contains enough LCL values that can be used. If so is not the case, then the function ends in an operation 599.

If so is the case, then in an operation 540, a table ALCL[ ] receives the LCL values that can be used thanks to the execution of a function Sel( ). Afterwards, in an operation 545 identical to the operation 515, the computer 12 executes the function Calc( ) which determines an agglomerated LCL value ALCL and a value CV for the LCL values. Alternatively, a function Calc2( ) may be applied which segments the table ALCL[ ] into two groups, and, if the centre of the two groups are separated by more than 15%, returns a value derived from the average or the median or otherwise barycentric of the group whose values are the highest.

Afterwards, the value CV is tested in an operation 550 by a Function CV( ) which determines two conditions in the example described herein:
the first condition relates to the fact that the value CV should be lower than 0.3, and
the second condition verifies that the value ALCL has not more than 50% difference with the immediately previous value ALCL.

The first condition relates to the consistence of the LCL values with one another, given the fact that they are close local measurements. The second condition is of an operational nature and aims to avoid sending information that might appear to be contradictory to the practitioner.

If one of these two conditions is not met, then it is considered that the value ALCL is unusable, and it is set to 0 in an operation 555. Otherwise, an estimate of the agglomerated LCL value has been calculated successfully and could be returned with all of the LCL values of the corresponding electrodes.

1. A device for determining a cardiac activation cycle length comprising a memory arranged so as to store electrogram data having time stamps and associated with a channel, a preparer arranged so as to receive electrogram data associated with a given channel and with a time window of at least 1.5 seconds, to extract therefrom baseline noise and high-frequency noise and to provide pre-processed data, a detector arranged so as to receive the pre-processed data and to detect therein non-overlapping activation segments that each correspond to a window within said time window of at least 1.5 seconds, which detector operates by determining local extrema in the pre-processed data and by grouping them into activation segments, and a computer arranged so as to determine a periodicity condition of the activations by determining in each activation segment a reference time point and by comparing the duration of intervals each defined by two reference time points consecutive to the duration of the time window of at least 1.5 seconds, and, in case of a periodicity condition indicating periodic activation segments, to determine an activation cycle length from the average or the median of the interval durations,
the detector being arranged so as to perform a post-processing by segmenting the intervals into three groups according to their duration, by searching for local extrema in the electrogram data corresponding to the intervals of the group of longest durations, and, where appropriate, by completing the activation segments with activation segments derived from these extrema.

2. The device according to claim 1, wherein the computer is further arranged so as to determine the periodicity condition of the activations from the ratio of the difference between the longest interval duration and the shortest interval duration divided by the average of the duration of the intervals.

3. The device according to claim 1, wherein the detector is arranged so as to calculate the ratio of the difference between the longest interval duration and the shortest interval duration divided by the average of the duration of the intervals, and to redetect the activation segments by modifying the detection of the extrema when this value exceeds a selected threshold.

4. The device according to claim 1, wherein the detector is arranged so as to perform a post-processing by cutting into two or more the activation segments whose duration is longer than twice the average of the durations of the activation segments.

5. (canceled)

6. The device according to claim 1, wherein the detector is arranged so as to perform post-processing by segmenting the intervals into three groups according to their duration, and by merging the activations segments whose reference time points define the intervals of the group of shortest durations.

7. The device according to claim 4, wherein the detector is arranged so as to calculate the ratio of the difference between the longest interval duration and the shortest interval duration divided by the average intervals of the intervals before and after post-processing, and to apply the post-processing only when this ratio decreases.

8. The device according to claim 1, wherein the computer is arranged so as to determine an agglomerated cycle length value from the cycle length value of several electrodes with reference to the same time window.

9. A method for determining a cardiac activation cycle length comprising:
a. receiving electrogram data having time stamps and associated with a given channel and with a time window of at least 1.5 seconds,
b. extracting from the electrogram data of the operation a) baseline noise and high-frequency noise to provide pre-processed data,
c. detecting in the pre-processed data non-overlapping activation segments each corresponding to a window within said time window of at least 1.5 seconds, by determining local extrema in the pre-processed data and grouping them into activation segments,
d. performing a post-processing by segmenting the intervals into three groups according to their duration, by searching for local extrema in the electrogram data corresponding to the intervals of the group of longest durations, and, where appropriate, by completing the activation segments with activation segments derived from these extrema,
e. determining a periodicity condition of the activations by determining in each activation segment a reference time point and by comparing the duration of intervals each defined by two consecutive reference time points with the duration of the time window of at least 1.5 seconds, and, in case of a periodicity condition indicating periodic activation segments, determining an activation cycle length from the average or the median of the interval durations.

10. The method according to claim 9, wherein the operation d) further comprises determining the periodicity condition of the activations from the ratio of the difference between the longest interval duration and the shortest interval duration divided by the average of the duration of the intervals.

11. The method according to claim 9, wherein the operation c) comprises c1) calculating the ratio of the difference between the longest interval duration and the shortest interval duration divided by the average of the duration of the intervals, and c2) re-detecting the activation segments while modifying the detection of the extrema when this value exceeds a selected threshold.

12. The method according to claim 9, wherein the operation c) comprises one or more of the following post-processing operations:
c3) cutting into two or more than two the activation segments whose duration is longer than twice the average of the durations of the activation segments,
c4) segmenting the intervals into three groups according to their duration, by searching for local extrema in the electrogram data corresponding to the intervals of the group of longest durations, and, where appropriate, by supplementing the activation segments with activation segments derived from these extrema,
c5) segmenting the intervals into three groups according to their duration, and by merging the activation segments whose reference time points define the intervals of the group of shortest durations.

13. The method according to claim 12, wherein the operation c) further comprises c6) calculating the ratio of the difference between the longest interval duration and the shortest interval duration divided by the average duration of the intervals before and after post-processing, and applying the post-processing only when this ratio decreases.

14. A computer program comprising instructions for executing the method according to claim 10 when said computer program is implemented by a computer.

15. A computer-readable data storage medium on which the computer program of claim 14 is recorded.
